(19) United States
(12) Patent Application Publication
(10) Pub. No.: US 2015/0359538 A1
(43) Pub. Date: Dec. 17, 2015

(54) COMPRESSIBLE STRUCTURE CLAMPING AND CUTTING DEVICE

(71) Applicant: New Life Innovations LLC, Yorktown, VA (US)

(72) Inventor: Stanley D. Yeatts, II, Yorktown, VA (US)

(21) Appl. No.: 14/738,985

(22) Filed: Jun. 15, 2015

Related U.S. Application Data
(60) Provisional application No. 62/013,046, filed on Jun. 17, 2014.

(51) Int. Cl. A61B 17/122; A61B 17/128

(52) U.S. Cl. CPC A61B 17/122; A61B 17/128; A61B 2017/1225

(57) ABSTRACT

A device for clamping and severing a compressible structure includes a clamp assembly and a cutting assembly. The clamp assembly has first second identical clamps coupled together to define a gap therebetween with posts spanning the gap. Each clamp has a pair of arms hingedly coupled to one another at first ends thereof and lockable to one another at second ends thereof. The clamp assembly defines an open position when the second ends of the arms are separated from one another and a clamped position when the second ends of the arms are locked to one another. The cutting assembly supports the clamp assembly in its open position and places the clamp assembly in its clamped position. The cutting assembly includes a convexly-curved cutting blade that passes through the clamp assembly's gap when the clamp assembly is in its closed position to cut through the posts spanning the gap.

Pursuant to 35 U.S.C. §119, the benefit of priority from provisional application 62/013,046, with a filing date of Jun. 17, 2014, is claimed for this non-provisional application.

### FIELD OF THE INVENTION

The invention relates generally to clamping and cutting devices, and more particularly to a device and method for clamping and cutting a compressible structure such as an umbilical cord.

### BACKGROUND OF THE INVENTION

The severing of a fluid-filled compressible structure typically involves fluid loss from one or both sides of the severed structure. In most instances, the fluid loss is not desirable. Accordingly, some form of clamping of the structure takes place prior to the severing thereof. One example of this is the umbilical cord of a newly-delivered baby that must be severed shortly after delivery. For years, this process has been accomplished by clamping the cord at two separated positions therealong. The physician, father or other attendant then cuts the cord between the two clamps. The limitations or drawbacks of this process are related to the accuracy, reliability, efficiency and hygiene of the process.

Using two separate clamps, three separate motions are required to clamp (twice) and then sever the umbilical cord. Further, a separate instrument must be handled by each motion thereby increasing the number of instruments that must be accessible on an instrument tray. Each separate motion requires valuable seconds at a time when even one or two seconds could be critical to the newborn's well-being. The clamped umbilical cord segment between the two separated clamps is under increased hydrostatic pressure. Thus, regardless of how small the gap there is between the two individually placed clamps, blood can be sprayed indiscriminately when the cord is cut. With today's concerns over blood-borne pathogens (e.g., HIV, Hepatitis viruses, etc.), even a little blood discharge can be a problem for physicians, nurses and all other people present at the delivery. Environmental hygiene is also compromised by unwanted blood droplets from cord transection reaching unprotected individuals and/or room surfaces.

In order to improve the process of cutting the umbilical cord, a variety of clamping and cutting devices have been developed. For example, in U.S. Pat. No. 4,716,886, a hand-held device includes two clamps that are held together in a side-by-side relationship by a shear pin. A cutting blade located between the two clamps is used to cut the cord clamped by the two clamps. The single device clamps and cuts with the same motion thereby addressing the convenience and speed issues outlined above. However, hygiene is still a concern as the gap between the two clamps contains blood under pressure that will be discharged when the cord is cut.

In U.S. Pat. No. 5,584,840, a similar structure to that just described is disclosed. An additional feature includes splash guards to prevent blood from splashing in the direction of surrounding individuals. While the hygiene concern is addressed in that the indiscriminate spraying of blood is prevented, the fact remains that blood will be discharged when the umbilical cord is cut.

More recently, U.S. Pat. No. 5,968,054 disclosed a device and method for clamping and severing a compressible structure containing a fluid. A clamp having a pair of arms is securable in a clamped position about a compressible structure containing a fluid so that the fluid between the arms is forced outward from the clamp. A cutting assembly coupled to the clamp cuts through the pair of arms in their clamped position to form first and second separated clamps that remain in the clamped position. As a result, the compressible structure is severed to form first and second severed ends thereof clamped by a respective one of the first and second separated clamps. While this device greatly advanced the state of the art, some instances of fluid escape occurred due to clamping or cutting inefficiencies of the clamp and/or cutting assembly. Further, the clamp in its pre-use state is a complex part requiring the use of costly molding techniques.

### SUMMARY OF THE INVENTION

Accordingly, it is an object of the present invention to provide a device and method for clamping and cutting a compressible structure such as an umbilical cord.

Another object of the present invention is to provide a device and method for clamping and cutting a compressible structure containing a fluid that prevents the substantial or consequential discharge of any of the fluid as the compressible structure is cut.

Still another object of the present invention is to provide a severable clamp that is readily producible.

Other objects and advantages of the present invention will become more obvious hereinafter in the specification and drawings.

In accordance with the present invention, a device for clamping and severing a compressible structure includes a clamp assembly and a cutting assembly. The clamp assembly has a first clamp and a second clamp coupled together to define a gap therebetween. The first clamp is identical to the second clamp. Each of the first clamp and second clamp has a pair of arms hingedly coupled to one another at first ends thereof and lockable to one another at second ends thereof. The first clamp is coupled to the second clamp with a plurality of posts spanning the gap therebetween. The clamp assembly defines an open position when the second ends of the arms are separated from one another and a clamped position when the second ends of the arms are locked to one another. The cutting assembly supports the clamp assembly in its open position and places the clamp assembly in its clamped position. The cutting assembly includes a convexly-curved cutting blade for passing through the clamp assembly's gap when the clamp assembly is in its closed position to thereby cut through the posts spanning the gap.

### BRIEF DESCRIPTION OF THE DRAWINGS

Other objects, features and advantages of the present invention will become apparent upon reference to the following description of the preferred embodiments and to the drawings, wherein corresponding reference characters indicate corresponding parts throughout the several views of the drawings and wherein:

FIG. 1 is a perspective view of the clamping and cutting device in its open or unclamped position in accordance with an embodiment of the present invention;

FIG. 2A is an isolated perspective view of a clamp assembly in its open or unclamped position in accordance with any embodiment of the present invention;

FIG. 2B is a side view of one of the two clamps used to construct a clamp assembly;

FIG. 2C is a cross-sectional view of one clamp arm taken along line 2C-2C in FIG. 2B;

FIG. 2D is a cross-sectional view of the other clamp arm taken along line 2D-2D in FIG. 2B;

FIG. 2E is an end view of a portion of the clamp assembly taken along line 2E-2E in FIG. 2A;

FIG. 3A is an isolated perspective view of the jaw assembly in its open position without the clamp assembly installed therein in accordance with an embodiment of the present invention;

FIG. 3B is an isolated perspective view of the jaw assembly shown in FIG. 3A in its closed position;

FIG. 4 is a perspective view of the two clamps after they have been severed from their assembled and clamped configuration to define first and second separated clamps that remain clamped onto the severed ends of the compressible structure such as an umbilical cord; and

FIG. 5 is a perspective view of another embodiment of a clamp assembly that can be used in the present invention.

### DETAILED DESCRIPTION OF THE INVENTION

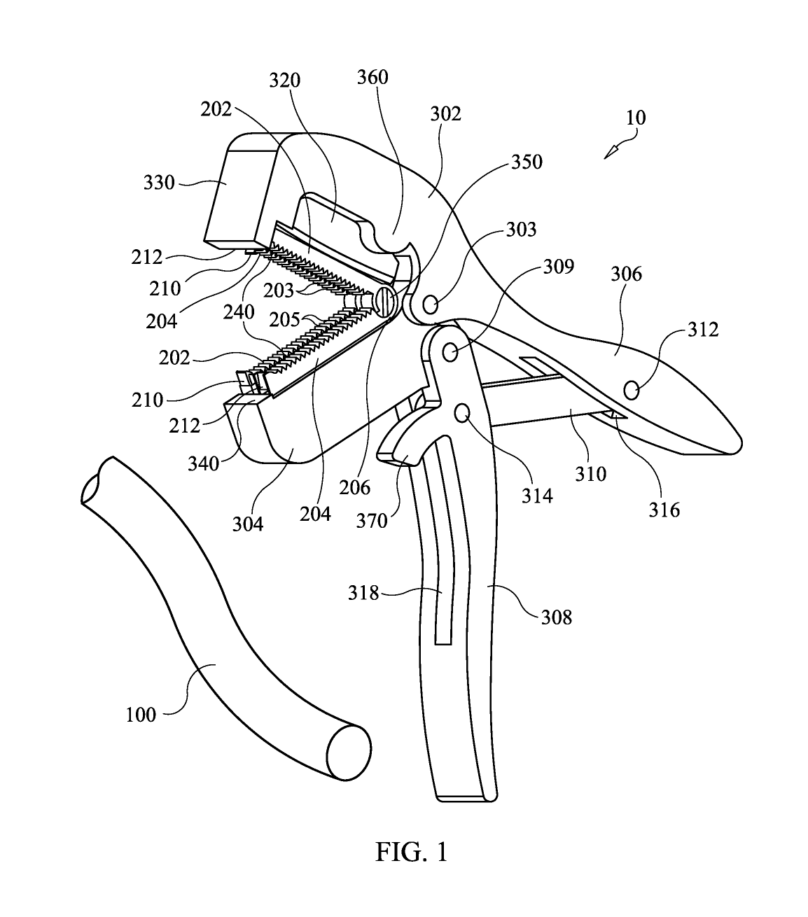

Referring now to the drawings, an embodiment of a clamping and cutting device in accordance with the present invention will be described with simultaneous reference to FIGS. 1-4. The same reference numerals will be used when describing common elements in the various drawings. The present invention can be used to clamp and cut a compressible structure while minimizing or eliminating any fluid discharge from the severed ends of the compressible structure. By way of example, the present invention will be described for its use in the clamping and cutting of an umbilical cord. However, the present invention could also be used to clamp and cut a variety of fluid-filled compressible structures or hoses used in medical, veterinary, biochemical or industrial applications.

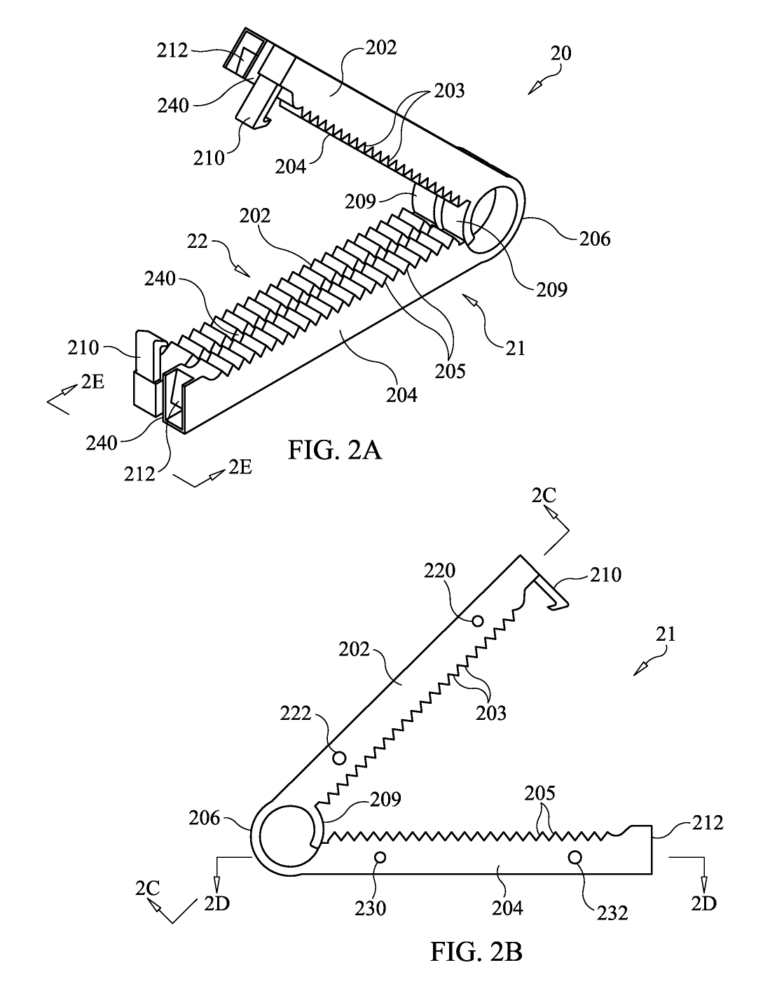

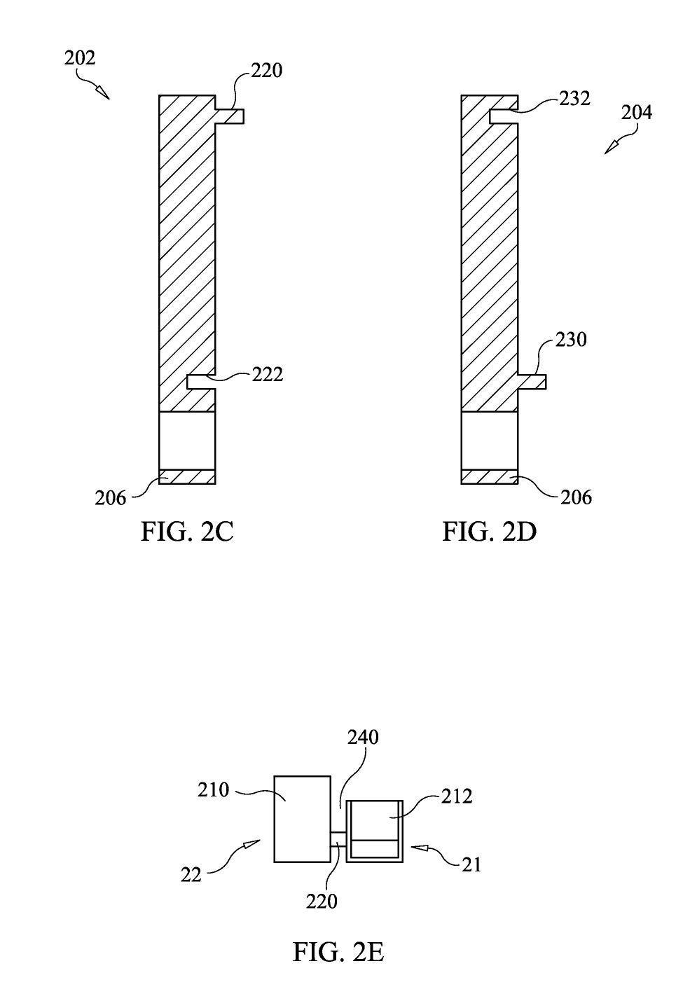

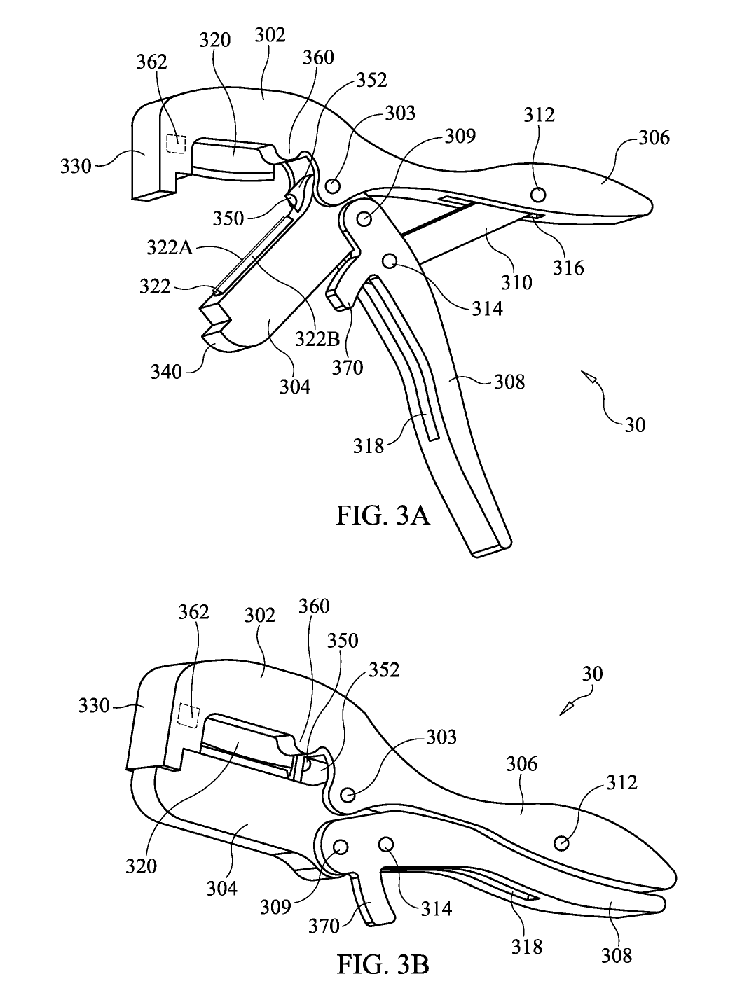

The entirety of the clamping and cutting device of the present invention is illustrated in FIG. 1 and referenced generally by numeral 10. In the illustrated embodiment, device 10 includes a clamp assembly 20 that is shown in isolation in FIG. 2A. Briefly, clamp assembly 20 is constructed from two identical clamps (one of which is illustrated in FIG. 2B and referenced generally by numeral 21) that are joined together and then severed by the present invention to form two separate clamps 21 and 22 as shown in FIG. 4. A hand held/gripped jaw assembly 30 used to sever clamp assembly 20 is shown in isolation in FIGS. 3A and 3B.

Materials used for clamp assembly 20 and jaw assembly 30 are preferably strong and substantially rigid to be capable of transmitting sufficient force through jaw assembly 30 and clamp assembly 20 in order to: i) clamp a compressible structure such as an umbilical cord 100, ii) cut cleanly through clamp assembly 20, and iii) cut cleanly through the compressible structure such as umbilical cord 100 thereby leaving both severed ends thereof clamped in clamps 21 and 22 as shown in FIG. 4. For example, most of the elements of clamp assembly 20 and jaw assembly 30 can be fabricated from polypropylene by injection molding or other suitable fabrication processes. While clamp assembly 20 would be made for a single use, jaw assembly 30 can be manufactured to be a single or multiple use component. In terms of its use in the cutting of an umbilical cord, clamp assembly 20 and jaw assembly 30 must be sterilized prior to use in ways well known in the art.

Referring to FIGS. 2A and 2B, clamp assembly 20 (FIG. 2A) is constructed from two identical clamps 21 (FIG. 2B) and 22. Accordingly, the details of clamp 21 illustrated in FIG. 2B will be the same for clamp 22 such that only clamp 21 will be described herein. FIG. 2B illustrates clamp 21 in a side view thereof to show details that will be located at what will be the central region of clamp assembly 20. Clamp 21 has a pair of arms 202 and 204 that can be secured in a clamped position. In the illustrated embodiments, arms 202 and 204 are hinged to one another at one end of clamp 21 by a hinge 206 which can be integral with arms 202 and 204. Hinge 206 can also be constructed to include a limit or stop tang 209, the presence of which prevents a compressible structure (that is to be clamped) from being pushed into and interfering with hinge 206. At the outboard ends of clamp 21, a latch is provided so that clamp 21 can be locked into a clamped position. More specifically, arm 202 has a latching tang 210 integral therewith while arm 204 has a latching seat 212 integral therewith. In use and as will be explained further below, arms 202 and 204 are pivoted (about hinge 206) towards one another until tang 210 latches or locks into seat 212.

Between the latch elements of clamp 21 (i.e., latching tang 210 and latching seat 212) and hinge 206, the opposing faces of arms 202 and 204 define clamping surfaces that can have a series of ridges 203 and 205, respectively, formed thereon such that ridges 203 interlock with ridges 205 when clamp 21 assumes its clamped position (FIG. 4). In the illustrated embodiment, ridges 203 and 205 are arranged generally parallel to one another and generally parallel to the compressible structure (not shown in FIGS. 2A and 2B for sake of clarity) that will be clamped thereby. Ridges 203 and 205 span the entire width of arms 202 and 204, respectively. Ridges 203 and 205 should cover an area between the hinge and latch portions of clamp 21 at least equal to the width of the compressible structure being clamped and typically would be provided over the entire length of arms 202 and 204 between the latch and hinge portions of clamp 21.

Referring now to FIGS. 2B-2D, the same side of each arm 202/204 is constructed to define one post and one hole. More specifically, arm 202 defines a post 220 near latching tang 210 and a hole 222 near hinge 206. Arm 204 defines a post 230 near hinge 206 and a hole 232 near latching seat 212. The position of the post and hole on each of arms 202 and 204 can be reversed without departing from the scope of the present invention. Further, the number of post-hole combinations used to construct a clamp assembly can be altered without departing from the scope of the present invention. Hole 222 is sized to receive a post 230 from another clamp (i.e., clamp 22), while hole 232 is sized to receive a post 220 from another clamp (i.e., clamp 22). The cross-sectional geometric shape of the posts and holes can be round (as shown) or other geometric shapes without departing from the scope of the present invention. Since clamps 21 and 22 are identical, the mating/assembly of two clamps 21 and 22 is achieved by orienting clamps 21 and 22 such that the sides of arms 202 and204 associated with clamp 21 oppose the sides of arms 204 and 202, respectively, of clamp 22 with the two hinges 206 being adjacent to one another as best shown in FIG. 2A. By doing so, each post on clamp 21 is aligned with a hole on clamp 22 and vice versa, and clamp assembly 20 is formed/defined when the posts and holes are pressed into engagement with each other. Clamp assembly 20 is thus defined by tang 210 of clamp 21 being adjacent seat 212 of clamp 22, while seat 212 of clamp 21 is adjacent tang 210 of clamp 22 as is clearly shown in FIG. 2A.

The above-described post-to-hole mating is designed such that, when clamp assembly 20 is created by the mating of clamps 21 and 22, a small gap 240 (FIG. 2A) is defined between clamps 21 and 22 with a portion of posts 220 and 230 spanning gap 240 as shown in FIG. 2E where a portion of post 220can be seen spanning gap 240. Although not visible in FIG. 2E, it is to be understood that a portion of post 230 also spans gap 240 in a spaced-apart alignment with post 220. Posts 220/230 and holes 222/232 can be designed for a snug fit, snap fit, etc., to retain their fitted relationship in clamp assembly 20. The post-to-hole coupling relationship is such that gap 240 is sized to define a sliding fit with a cutting blade that is to pass through as will be described below.

Jaw assembly 30 is shown as part of device 10 in FIG. 1, and is shown in isolation (i.e., no clamp assembly 20 is shown for clarity of illustration) in its open position in FIG. 3A and in its closed position in FIG. 3B. Briefly, jaw assembly 30 places clamp assembly 20 in its clamped position about the periphery of a compressible structure, cuts through clamp assembly 20 and the compressible structure, and releases the severed portions of clamp assembly 20 in the form of clamps 21 and 22.

In the illustrated example, jaw assembly 30 includes an upper jaw 302 coupled to an upper handle 306 and a lower jaw 304 coupled to a lower handle 308. Jaws 302 and 304 are hinged to one another at hinge pin 303. Upper handle 306 is fixed to or made integral with jaw 302. Lower handle 308 is pivotally coupled to jaw 304 by hinge pin 309 located beneath hinge pin 303. Upper handle 306 and lower handle 308 are coupled to one another by a leverage link 310. More specifically, leverage link 310 is pivotally coupled to upper handle 306 by hinge pin 312 and to lower handle 308 by hinge pin 314. To allow handles 306 and 308 to be squeezed together, slots 316 and 318 are provided in upper handle 306 and lower handle 308, respectively. The mechanical advantages offered by this design of jaw assembly 30 include: i) jaws 302 and 304 remain open until handles 306 and 308 are squeezed together; ii) during the initial movement of handle 308, a relatively quick closure of jaw 304 towards jaw 302 is achieved with only a small amount of movement of handle 308; iii) during later movement of handle 308, a relatively slow closure but with high force being generated between jaws 304 and 302 is achieved to cleanly sever the clamp assembly; and iv) better control over the clamping operation while requiring less force than if the clamp assembly were just squeezed together by hand.

The opposing portions of jaws 302 and 304 cooperate with elements of clamp assembly 20 to seat clamp assembly 20 therein in both its open and clamped positions, and to release severed clamps 21 and 22 therefrom after they (and the clamped compressible structure) have been severed. A convexly-curved cutting blade 320 (e.g., a metal blade) is attachable or fixed therealong to jaw 302. A longitudinally split positioning anvil 322 is attachable to or fixed therealong to jaw 304. Blade 320 and anvil 322 are positioned such that when jaws 302 and 304 are closed as shown in FIG. 3B, blade 320 will come to rest between the opposing sides 322A and 322B of anvil 322.

Jaw assembly 30 can include several elements that help position/retain clamp assembly 20 in jaws 302/304 and aid in the closing of clamp assembly 20 when jaws 302/304 are closed together. In terms of positioning/retaining clamp assembly 20 in jaws 302/304, the outboard end of jaw 302 can define a hood 330 covering the end of cutting blade 320 and defining a seat for portions of the outboard ends of one arm of each of clamps 21 and 22. The outboard end of jaw 304 can define a lip 340 that forms a seat for portions of the outboard ends of the other arm of each of clamps 21 and 22. Jaw 304 also has a semicircular pin 350 extending from either side of a mounting plate 352 coupled to jaw 304 near hinge pin 306. Each pin 350 fits into a portion of hinge 206 of clamps 21 and 22. In terms of aiding the closing of clamp assembly 20, jaw 302 has a boss 360 integrated therewith on either side of blade 320. Boss 360 is sized/shaped to apply pressure to each hinge 206 (of clamps 21 and 22) when jaws 302/304 are closed together. In this way, a clamping force is applied at the point of greatest resistance to the closure of clamps 21 and 22 as jaws 302/304 are drawn together. At the outboard end of jaw 302, a second boss 362 is integrated therewith on either side of blade 320. Boss 362 is within hood 330 and is sized/shaped to apply pressure to the outboard end of clamp assembly 20 as jaws 302/304 are in the final phase of being closed together. In this way, the outboard end of clamp assembly 20 is stabilized while closing pressure is applied to assure the mating of tangs 210 and seats 212. To improve one's leverage when closing jaws 302/304, handle 308 can include an integrated finger rest 370 for cradling one's index finger when jaw assembly 30 is grasped. Finger rest 370 is positioned on handle 308 to encourage proper hand placement for optimal leverage when squeezing jaw assembly 30.

In operation, with jaws 302 and 304 in their open position as shown in FIG. 3A, clamp assembly 20 is seated therein by placing clamp 21 on one side of jaws 302/304 and then attaching clamp 22 to clamp 21 from the other side of jaws 302/304. The attachment of clamps 21/22 to one another is made via the above-described post/hole coupling. In this open position, cutting blade 320 resides above the posts 220/230 spanning gap 240 of clamp assembly 20 (FIG. 1). The combination of hood 330, lip 340, and pins 350 position and retain clamp assembly 20 in place between jaws 302/304. The assembly of clamp assembly 20 in its open (pre-use) position for support by jaw assembly 30 can be accomplished in a factory setting or just prior to use without departing from the scope of the present invention.

The compressible structure (e.g., umbilical cord 100) to be severed is placed between arms 202 and 204. Handles 306 and 308 are then squeezed by an individual applying a gripping force. In general, as handles 306 and 308 are squeezed, jaws 302 and 304 apply a compressive force to arms 202 and 204 until latching tang 210 seats in a respective latching seat 212 thereby locking arms 202 and 204 together for each of clamps 21 and 22. At this point, clamp assembly 20 is in its clamped and locked position with ridges 203 and 205 tending to interlock with umbilical cord 100 being fully compressed therebetween. The portion of umbilical cord 100 passing through clamp assembly 20 has its contained blood and/or other fluids (not shown) pushed outward to either side of clamps 21 and 22. More specifically, as handles 306 and 308 are continually squeezed together, curved blade 320 first cuts through post 230 that spans gap 240 nearest hinge 206, then umbilical cord 100, and finally through post 220 nearest tangs 210 and seats 212. That is, the convex curve of blade 320 allows the cutting process to proceed sequentially from the point nearest hinge 206 to the point nearest the outboard ends of clamps 21 and 22. The above-described sequential cutting method/technique is facilitated by the curved blade and/or others of the above-described features that allow the compressible structure (e.g., umbilical cord 100) to be secured by the engagement of the tangs/seats prior to completion of the cutting of the clamp assembly into two functional clamps, while also slowly releasing the pressure in umbilical cord 100 to thereby minimize any spurting fluid. Bosses 360 and 362 provide stabilizing and pressure forces to clamp assembly 20 to minimize the squeezing force needed and assure tang-to-seat engagement for each of the two functional clamps 21 and 22.

Once severed from their clamping assembly relationship, clamps 21 and 22 are free to move laterally away from jaw assembly 30 (when jaws 302/304 are opened) as shown in FIG. 4. That is, clamp 21 remains clamped, via its latching tang 210 and latching seat 212, onto one severed end of umbilical cord 100. Similarly clamp 22 remains clamped, via its latching tang 210 and latching seat 212, onto the other severed end of umbilical cord 100. The severed portions of posts 220 and 230 from clamp 21 that remain in respective holes 232 and 222 of clamp 22 are referenced by numerals 220₂₁ and 230₂₁. Since there is little or no fluid present in the severed portion of umbilical cord 100, no substantial or consequential amount of fluid will be discharged into the surrounding environment.

The advantages of the present invention are numerous. The device allows a fluid-filled compressible structure to be severed without consequential fluid discharge. Further, the clamping and cutting operations are conveniently accomplished with a single motion thereby allowing the operation to be performed quickly. The device can be made as a completely disposable or reusable item for use in medical, veterinary, or industrial procedures. In the illustrated embodiment, the jaw assembly need not be oriented for either left or right-handed individuals and is therefore a multi-directional tool. The device's simplicity of operation means that even a non-professional could use the device effectively. The simple clamp design facilitates creation of the clamp assembly, simplifies manufacturing, and reduces costs. The jaw assembly can include a number of features that improve the device's clamping and cutting efficiency.

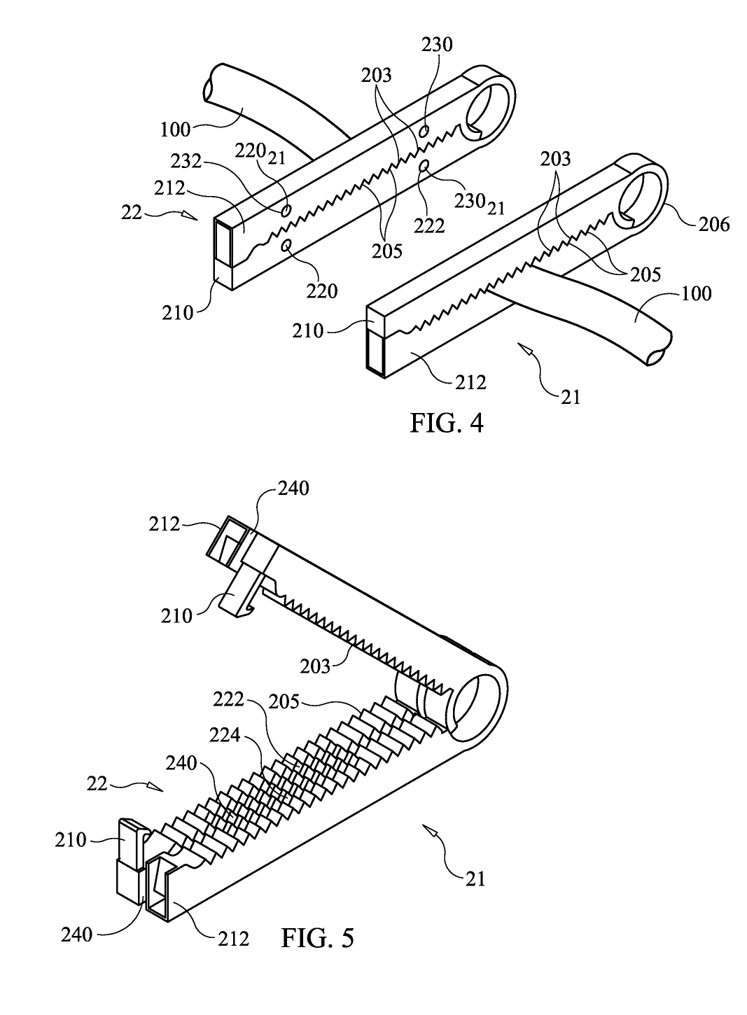

Although the invention has been described relative to a specific embodiment thereof, there are numerous variations and modifications that will be readily apparent to those skilled in the art in light of the above teachings. For example, ridges 203 and 205 could be eliminated or replaced with any type of serration, teeth or other structure as dictated by the particular application. Another possibility is to provide grooves that cut across the ridges in order to prevent a compressible structure from slipping through the clamp. This is illustrated in FIG. 5 where a groove 222 is cut perpendicularly across ridges 203 and 205 in each of clamps 21 and 22 of clamp assembly 20 that will be severed. In this way, as clamp assembly 20 is placed in its clamped position, the compressible structure is captured in each groove 222 to prevent it from slipping in a direction perpendicular thereto. It is therefore to be understood that, within the scope of the appended claims, the invention may be practiced other than as specifically described.

What is claimed as new and desired to be secured by Letters Patent of the United States is:

1. A device for clamping and severing a compressible structure, comprising:
a clamp assembly having a first clamp and a second clamp coupled together to define a gap therebetween, said first clamp being identical to said second clamp, each of said first clamp and said second clamp having a pair of arms hingedly coupled to one another at first ends thereof and lockable to one another at second ends thereof, each of said first clamp and said second clamp having one side thereof defining a plurality of posts and a plurality of holes wherein, in said clamp assembly, (i) each one of said posts from said first clamp engages one of said holes from said second clamp, and (ii) each one of said posts from said second clamp engages one of said holes from said first clamp, and wherein each of said posts spans said gap;
said clamp assembly defining an open position when said second ends of said arms are separated from one another, and defining a clamped position when said second ends of said arms are locked to one another; and
a cutting assembly for supporting said clamp assembly in said open position thereof and for placing said clamp assembly in said clamped position thereof, said cutting assembly including a convexly-curved cutting blade for passing through said gap when said clamp assembly is in said closed position and for cutting through said posts spanning said gap.

2. A device as in claim 1 wherein, for each of said first clamp and said second clamp, said plurality of posts comprises two posts and said plurality of holes comprises two holes.

3. A device as in claim 2, wherein one of said two posts and one of said two holes is provided on each arm associated with each said pair of arms.

4. A device as in claim 1, wherein each said pair of arms defines ridges that interlock when said clamp assembly is in said clamped position.

5. A device as in claim 1, wherein said cutting assembly includes opposing jaws for supporting said clamp assembly in said open position.

6. A device as in claim 5, further comprising handles coupled to said jaws for use in drawing said jaws together wherein said clamp assembly is placed in said clamped position and said posts are cut.

7. A device as in claim 5, further comprising:
pin elements coupled to said jaws for supporting each of said pair of arms at said first ends thereof; and
seating elements coupled to said jaws for receiving portions of each of said pair of arms at said second ends thereof.

8. A device for clamping and severing a compressible structure, comprising:
a clamp assembly having a first clamp and a second clamp coupled together to define a gap therebetween, said first clamp being identical to said second clamp, each of said first clamp and said second clamp having a pair of arms hingedly coupled to one another at first ends thereof and lockable to one another at second ends thereof, said first clamp being coupled to said second clamp wherein a plurality of posts spans said gap;
said clamp assembly defining an open position when said second ends of said arms are separated from one another, and defining a clamped position when said second ends of said arms are locked to one another; and
a cutting assembly for supporting said clamp assembly in said open position thereof and for placing said clamp assembly in said clamped position thereof, said cutting assembly including a convexly-curved cutting blade for passing through said gap when said clamp assembly is in said closed position and for cutting through said posts spanning said gap.

9. A device as in claim 8 wherein said plurality of posts comprises two posts.

10. A device as in claim 8, wherein each said pair of arms defines ridges that interlock when said clamp assembly is in said clamped position.

11. A device as in claim 8, wherein said cutting assembly includes opposing jaws for supporting said clamp assembly in said open position.

12. A device as in claim 11, further comprising handles coupled to said jaws for use in drawing said jaws together wherein said clamp assembly is placed in said clamped position and said posts are cut.

13. A device as in claim 11, further comprising:
pin elements coupled to said jaws for supporting each of said pair of arms at said first ends thereof; and
seating elements coupled to said jaws for receiving portions of each of said pair of arms at said second ends thereof.

14. A device for clamping and severing a compressible structure, comprising:
a clamp assembly having a first clamp and a second clamp coupled together to define a gap therebetween, said first clamp being identical to said second clamp, each of said first clamp and said second clamp having a pair of arms hingedly coupled to one another at first ends thereof and lockable to one another at second ends thereof, said first clamp being coupled to said second clamp wherein a plurality of posts spans said gap;
said clamp assembly defining an open position when said second ends of said arms are separated from one another, and defining a clamped position when said second ends of said arms are locked to one another; and
a cutting assembly having jaws for supporting said clamp assembly in said open position thereof when said jaws are opened, one of said jaws having a convexly-curved cutting blade mounted thereon and aligned with said gap, said cutting assembly further having handles coupled to said jaws for bringing said jaws together wherein said clamp assembly is placed in said clamped position and said cutting blade subsequently cuts through said posts.

15. A device as in claim 14 wherein said plurality of posts comprises two posts.

16. A device as in claim 14, wherein each said pair of arms defines ridges that interlock when said clamp assembly is in said clamped position.

17. A device as in claim 14, further comprising:
pin elements coupled to said jaws for supporting each of said pair of arms at said first ends thereof; and
seating elements coupled to said jaws for receiving portions of each of said pair of arms at said second ends thereof.
